(19) United States
(12) Patent Application Publication
(10) Pub. No.: US 2024/0288420 A1
(43) Pub. Date: Aug. 29, 2024

(54) MASK-BASED DIAGNOSTIC DEVICE WITH ACCESSIBILITY FEATURES

(71) Applicant: John J. Daniels, Madison, CT (US)

(72) Inventor: John J. Daniels, Madison, CT (US)

(21) Appl. No.: 18/113,777

(22) Filed: Feb. 24, 2023

(51) Int. Cl. G01N 33/543; A61B 5/00; A61B 5/097; G01N 27/414

(52) U.S. Cl. CPC G01N 33/5438; A61B 5/097; A61B 5/7405; G01N 27/4141; G01N 27/4145

(57) ABSTRACT

A mask-based diagnostic device with accessibility features, includes a face mask, where an inside of the face mask defines during use a confined local environment that includes breath vapor exhaled from the lungs of a user. An exhaled breath condensate (EBC) collector has a condensate forming surface for converting exhaled breath vapor into an EBC liquid sample. An EBC testing unit tests the EBC sample for a target molecule, where the EBC sample contains water and the target molecule, the EBC testing unit includes a printed circuit board supporting a semiconductor packaged electronic biosensor in electrical communication with power, analysis and communications electronics, a fluid conductor for conducting the EBC sample to the electronic biosensor.

### BACKGROUND

The exemplary and non-limiting embodiments relate generally to diagnostic systems, methods, devices and computer programs and, more specifically, relate to digital and analog diagnostic devices for detecting a biomarker of a biological agent such as a coronavirus, lung cancer, tuberculosis, asthma, and other respiratory ailments and conditions, and/or blood borne biomarkers and other biomarkers that are present in the exhaled breath of a test subject.

The present invention also pertains to a device architecture, specific-use applications, and computer algorithms used to detect biometric parameters for the treatment and monitoring of physiological conditions in humans and animals by testing for biomarkers in exhaled breath, sweat, blood, urine, feces, gastro-intestinal lavage, interstitial fluid, mucus, saliva, or other bodily fluid.

This section is intended to provide a background or context to the exemplary embodiments of the invention as recited in the claims. The description herein may include concepts that could be pursued but are not necessarily ones that have been previously conceived, implemented or described.

Therefore, unless otherwise indicated herein, what is described in this section is not prior art to the description and claims in this application and is not admitted to being prior art by inclusion in this section.

Testing for biomarkers that indicate exposure, infection, progression and recovery from a disease condition, can be used to screen individuals for infection. Especially for diseases caused by novel pathogens like the recent SARS-CoV-2 virus, testing can help slow the spread of the virus. For example, protein and RNA testing for active virus shows who is currently contagious. Antibody testing can be used to find the members of a population that have recovered from the virus.

Diagnostics of an infection, such as SARS-CoV-2 infection, using real-time reverse-transcription polymerase chain reaction (RT-PCR) on nasopharyngeal swabs is now well-established, with saliva-based testing being lately more widely implemented for being more adapted for self-testing approaches. The procedure to obtain nasal swab samples is not only uncomfortable, but also often requires specialized personnel with risk of contaminating the person performing the test. Saliva tests have the advantage of being simpler to perform, less invasive with limited risks and RT-PCR on saliva specimens has becoming more widely implemented. The viscose nature of saliva together with the presence of saliva proteases, responsible for the proteolytic activity of saliva, make the direct application of saliva samples challenging. It is well known that the major mechanisms of COVID-19 spread are airborne and contact infections primarily due to aerosol droplets expelled from the lungs and airways of infected persons. There is therefore a growing need for sample collection by patients themselves and a simple to use testing system that can detect a target biomarker indicative of a pathogenic infection from a biosample obtained from the lungs and airways.

For individuals with disabilities, such as low vision, blindness, motor control and age-related difficulties, using a conventionally available home diagnostic system is often time a frustrating experience producing inaccurate test results due to the relatively complex sample taking and test procedures. There is therefore an established need for a simple diagnostic device that can be used by persons with disabilities without requiring any assistance.

### BRIEF SUMMARY

The below summary section is intended to be merely exemplary and non-limiting. The foregoing and other problems are overcome, and other advantages are realized, by the use of the exemplary embodiments of this invention.

In one aspect, a mask-based diagnostic (MBD) system for providing test results using an exhaled breath condensate (EBC) biosample in less than 10 minutes, includes: a mask having an EBC collector for converting exhaled breath vapor into a collected liquid biosample; a biosensor for generating an output signal dependent on the detection of a target biomarker in the liquid biosample; and electronics for determining a test result from the output signal and controlling the generation of audible spoken word messages for providing automatic readout of at least one of test instructions, test progress and the test results.

The MBD has “just put the mask on and breath” simplicity. Ideally suited for self-testing by a visually or physically challenged individual. The self-contained MBD non-invasively obtains an exhaled breath condensate (EBC) biosample, with test results available in less than 10 minutes. As an example, a tactile-locatable push button provides spoken word audio of test instructions, progress and results. The MBD eliminates need to collect a biosample, handle test components, mix materials, read or interpret visual indications.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an MBD with a self-contained user interface comprising a tactile-locatable pushbutton, a spoken word generating electronic circuit. and speaker and/or headphone jack include on the MBD.

FIG. 2 illustrates another version of the MBD with a Bluetooth wireless transmitter onboard the MBD for enabling a smartphone, smart speaker, accessibility device or wireless headphones to be used as the user interface for providing spoken word test instructions, progress and results to the user or test administrator.

FIG. 3 illustrates an assembled EBC testing unit.

FIG. 4 is an exploded view showing components of an EBC testing unit.

FIG. 5 illustrates a printed circuit board having a biosensor, fluid detectors, analysis and communications electronics and a battery.

FIG. 6 is a flow chart showing the use work flow for the MBD with accessibility features.

FIG. 7 is a schematic of the EBC testing unit with Bluetooth communication capability.

FIG. 8 is a block diagram showing some of the components onboard the MBD for providing a self-contained testing system with accessibility features.

FIG. 9 shows a smartphone having an application (APP) for communicating with the EBC testing unit and conveying information to the phone user and/or to a remote network server.

FIG. 10 shows screens of a smartphone APP providing a graphical user interface for conveying the steps for conducting a test using a mask-based diagnostic device and receiving information indicating the test results.

FIG. 11 illustrates a commercially available face mask with a retrofit EBC Collector having a collection pool.

FIG. 12 illustrates an unfolded face mask substrate suitable for roll-to-roll manufacturing and 2 supporting EBC collecting feature, fluid conductor, testing and communications components, where when folded and assembled, an MBD such as shown in FIG. 2 is provided.

FIG. 13 illustrates an embodiment of the mask-based diagnostic device having an EBC collector and sample collection pool that is retrofit into a pre-existing face mask;

FIG. 14 illustrates an EBC collector with a centrally mounted EBC testing unit.

FIG. 15 illustrates the EBC testing unit showing a dissolvable surfactant film disposed in the fluid flow path before the sample flow reaches the electronic biosensor.

FIG. 16 illustrates a version of the EBC testing unit showing two parallel biosensors for simultaneously receiving the fluid sample at the detection area of each biosensor.

FIG. 17 is an exploded view showing the components of the EBC collector.

FIG. 18 illustrates a manufacturing process for forming the EBC Collector.

FIG. 19 is an isolated view of a semiconductor double in-line packaged (DIP)biosensor for incorporation in a printed circuit board.

FIG. 20 illustrates the lid of a table top EBC testing unit.

FIG. 21 illustrates the housing in which sits an EBC testing unit printed circuit board (PCB).

FIG. 22 illustrates an assembled table top EBC testing unit showing a testing well for receiving a liquid sample.

FIG. 23 illustrates a single bare die wire bonded for semiconductor packaging and having multiple and differently functionalized g-FET biosensors.

FIG. 24 illustrates bare die biosensor having g-FET devices functionalized for FluA/FluB/SARS virus testing.

FIG. 25 illustrates a flow chart showing multiple data paths for test results transmission of trusted receiver, contract tracing and population studies.

### DETAILED DESCRIPTION

Below are provided further descriptions of various non-limiting, exemplary embodiments. The exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The word “exemplary” is used herein to mean “serving as an example, instance, or illustration.” Any embodiment described herein as “exemplary” is not necessarily to be construed as preferred or advantageous over other embodiments. In any case, all of the embodiments described in this Detailed Description are exemplary embodiments provided to enable persons skilled in the art to make or use the invention and not to limit the scope of the invention which is defined by the claims.

Many configurations, embodiments, methods of manufacture, algorithms, electronic circuits, microprocessors, memory and computer software product combinations, networking strategies, database structures and uses, and other aspects are disclosed herein for a diagnostic or testing platform, devices, methods and systems that have a number of medical and non-medical uses.

Although embodiments are described herein for detection of biomarkers of SARS-CoV-2 virus, the systems, methods and apparatus described are not limited to any particular virus or disease, or just limited to biological use-cases. In most instances, where the term virus or COVID-19 is used, any other health or fitness related biomarker could be used instead. The description here and the drawings and claims are therefore not intended to be limited in any way to virus detection, the inventions described and claimed can be used for many diseases and related biomarkers or target molecules/analytes including lung cancer, diabetes, asthma, tuberculosis, environmental exposures, glucose, lactate, blood borne diseases and other ailments or indications of the health of the test subject. Further, the electronic biosensor, test systems, uses and methods of manufacturing described herein are not limited to the use of exhaled breath condensate. Wastewater, potable water, environmental quality samples, ambient samples and any bodily fluid can be used as the test sample. The use of aptamers and engineered capture molecules, in particular, make the inventive sensor widely useful because of the nature of engineered capture molecules such as aptamers, nanoCLAMPs, nano-bodies, engineered anti-bodies, etc., being adaptable by specific engineering design and selection to have a binding affinity that is tailored to a corresponding target analyte. Therefore, the descriptions of innovations are not intended to be limited to a particular use-case, sample medium, capture molecule, biomarker or analyte.

In immunochromatography, a capture molecule, which may be, for example, an aptamer, nanoCLAMP, naturally occurring antibody, or engineered antibody, is disposed onto a surface of a porous membrane, and a sample passes along the membrane. As described herein, the term antibody, aptamer, engineered antibody, or capture molecule is used interchangeably. In some instances, a specific type of capture molecule may be described. In the case of a lateral flow assay (LFA) type testing system, biomarkers in the sample is bound by the capture molecule which is coupled to a detector reagent. As the sample passes through the area where the capture molecule is disposed, a biomarker detector reagent complex is trapped, and a color develops that is proportional to the concentration or amount biomarker present in the sample. In the case of an electronic or electro-chemical testing system, the captured biomarker causes a detectable change in a signal obtained, typically through an electrical connection with two or more electrodes.

The present invention relates to a mask-based diagnostic (MBD) with accessibility features that is suitable for self-testing by individuals with visual or physical impairments. The MBD provides PCR-comparable results using an exhaled breath condensate (EBC) biosample in less than 10 minutes. The MBD is an advancement of DiagMetrics' MBD that condenses exhaled breath vapor into a liquid, and it includes a proprietary g-FET biosensor that has excellent sensitivity and selectivity for detecting Covid-19.

The MBD is designed to provide simple and accessible usability for users with disabilities. The user only needs to wear the mask and breathe normally for about 10 minutes, which is the same as wearing a conventional face mask. The MBD includes electronics that generate visual and audible signals to provide automatic readout of the test results. The use of the MBD is as easy as putting on a conventional face mask, and the user can follow simple spoken instructions to perform the test.

The EBC biosample tested using a graphene-based biosensor offers synergistic advantages over all other available OTC and laboratory tests. The liquid biosample is mostly water and presents a very clean and immediate snapshot of the infection. The MBD's accessibility characteristics include the simplest of usability requirements and automatic readout in the form of visual and audible signals.

In one aspect, a mask-based diagnostic (MBD) system provides test results using an exhaled breath condensate (EBC) biosample in less than 10 minutes. The MBD 100 comprises a speaker 102, an EBC collector 104, a face mask 106, analysis electronics 108, and a biosensor 110. The EBC collector converts exhaled breath vapor into a collected liquid biosample; a biosensor for generating an output signal dependent on the detection of a target biomarker in the liquid biosample; and analysis electronics 108 for determining a test result from the output signal. A microcontroller controls the generation of audible spoken word messages for providing automatic readout of at least one of test instructions, test progress and the test results. A self-contained version of the MBD with accessibility features includes a voice generation circuit and speaker or headphone jack provided on the mask. The mask-based diagnostic device may also include the electronics and biosensor as components of an EBC testing unit for testing the liquid sample for the target biomarker, where the EBC sample contains water and may contain the target molecule. The EBC testing unit tests the EBC sample for the presence of the target molecule and includes the electronics formed on a printed circuit board supporting the biosensor. The biosensor is provided as a semiconductor packaged electronic biosensor in electrical communication with power, analysis and communications electronics. A fluid conductor, such as microfluidic channels comprising a suitable material for conducting the EBC sample to the electronic biosensor.

The MBD incorporates sensors that detect the flow of EBC over the biosensor. To use the MBD, the user just presses a tactile-locatable pushbutton. For example, on the first press of the push button a message is automatically played providing the general instructions for the test, including an instruction to press the push button with a long press to hear the instructions again or a short press to begin the test. Once the user has pressed the push button for a short duration (indicating an intention to start the test), the thermal mass of the EBC Collector is checked to see if the system is cold enough. The user puts on the mask and begins breathing, and in about 30 seconds, EBC begins to collect, and the start of the EBC flow is detected by a first liquid detection circuit. A spoken message from the speaker alerts the user that the test is in progress and to keep breathing normally. The EBC continues to flow over the biosensor until it reaches a second liquid detection circuit indicating a valid test. The biosensor and reader electronics analyze the EBC to determine if a Covid-19 biomarker is present.

After the test results are obtained, a spoken message alerts the user that a valid test has been performed, and the test results are either negative or positive, or if a problem has occurred, invalid. The test kit components are minimal, and the MBD is packaged in a resealable plastic bag with a sheet of paper containing simple written instructions and a QR code for access to a special accessibility-configured website. This version of the MBD with accessibility features is entirely self-contained, and the instructions tell the user to place the mask into the freezer for at least a half hour, then simply put the mask on and breathe normally for ten minutes. In the case of a visually impaired user that cannot read the instruction sheet, a family member or caregiver may tell the user one time the simple instructions, or the instructions can be provide via spoken word, braille, special accessibility device, smartphone, smart speaker, etc.

Another version of the MBD with accessibility features includes communications electronics 204 for wirelessly communicating with a smartphone 206, where the speaker 202 of the smart phone is used for playing the audible spoken word messages to the user for providing automatic readout of the test instructions, progress and results. In this case, a special QR code or spoken command can be used by an APP to facilitate pairing the MBD with the user's smartphone.

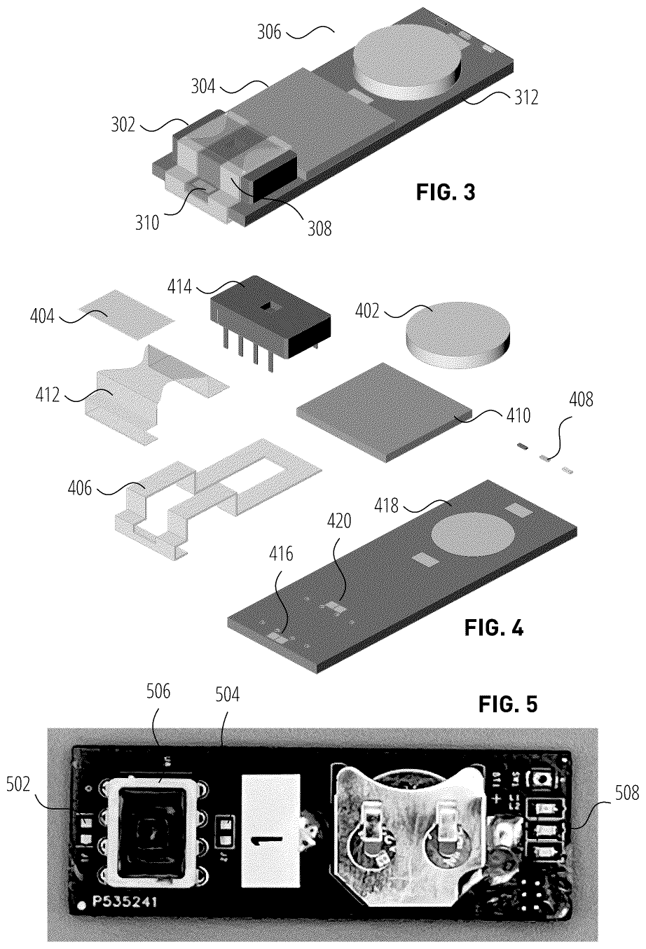

FIG. 3 illustrates an assembled EBC testing unit. The EBC testing unit includes the biosensor 302, the EBC testing unit 306, a wick 304, a fluid conductor 308, a fluid detector 310, and a printed circuit board 312. The fluid conductor conducts the EBC sample to the electronic biosensor. The mask-based diagnostic device may also include the electronics and biosensor as components of an EBC testing unit for testing the liquid sample for the target biomarker, where the EBC sample contains water and the target molecule. The EBC testing unit includes the electronics formed on a printed circuit board supporting the biosensor provided as a semiconductor packaged electronic biosensor in electrical communication with power, analysis and communications electronics. A fluid conductor, such as microfluidic channels comprising a suitable material are provided for conducting the EBC sample to the electronic biosensor. The mask-based diagnostic device may also include the electronics and biosensor as components of an EBC testing unit for testing the liquid sample for the target biomarker.

FIG. 4 is an exploded view showing components of an EBC testing unit 204. The EBC testing unit includes the biosensor 302, the wick 304, the fluid conductor 308, the fluid detector 310, the printed circuit board 312, a battery 402, a light emitting diode LED 408, a microfluidic cover 404, and a microfluidic adhesive 406. The fluid detector onboard the printed circuit board detects when EBC is flowing through the fluid conductor and into the detection well of the biosensor. As an example, the fluid detector comprises a pair of parallel conductors on the printed circuit board with a gap of about 0.02″ between the conductors that enables the detection of the presence of EBC in the section of the fluid conductor in contact with the parallel conductors. When EBC is present, an electrical signal can flow from one of the parallel conductors through the EBC to the other parallel conductor to complete an electrical circuit. In the embodiment shown in FIG. 4, two sets of fluid detectors are printed on the printed circuit board. Either or both the start of fluid conduction through the fluid conductor in a flow path before the electronic biosensor and the end of fluid conduction in the flow path after the electronic biosensor can be detected by the respective fluid detectors. The EBC testing unit also includes the printed circuit board supporting the semiconductor packaged electronic biosensor in electrical communication with power, analysis and communications electronics.

A transistor may be provided on the printed circuit board to amplify the signal generated when EBC is present in the fluid conductor section in contact with the parallel conductors. Alternatively, a TTL Logic circuit (e.g., High(3.3v) and Low(0v) can be the pads, and switch on LEDs to detect when the EBC biosample hits the first and second set of liquid detection pads. The fluid detectors can be used to determine the time it takes for the EBC biosample to flow from the first to second set of parallel conductors to indicate how well the EBC collector is performing at converting breath vapor to liquid. The fluid detectors can as be used to indicate EBC is present at the detection well of the biosensor and to automatically initiate the testing process and analysis of the electrical signals generated by the biosensor. In addition, or alternatively, a physical switch can be provided on the printed circuit board to initiate the testing, and/or a smartphone or computer user-interface wirelessly connected with the microcontroller or microprocessor onboard the PCB can be used to control the test. Also, the detection of the EBC at the second fluid detector (in the EBC flow path after the biosensor) can be used to indicate if the test is invalid, for example, if the EBC flow does not reach the second fluid detector.

The MBD system comprises a fluid detector 502, a fluid detector 504, a biosensor 506, and an LED 508. The mask-based diagnostic device may also include the fluid detector formed as a pair of adjacent electrical conductors in a fluid detection region of the flow path of the EBC liquid sample. A change in conductivity in a gap between the pair of conductors is detected when the EBC sample flows in the fluid detection region. As an example, the fluid conductor can comprise a strip of filter paper that absorbs and transfers the EBC liquid sample from the collection pool of the EBC Collector, flows the EBC liquid sample over a first fluid detector where the conductivity between the adjacent conductor changes because the presence of ions in the EBC liquid sample. This detection of the EBC flow is used to trigger, for example, a spoken word message that confirms to the user that EBC is being collected and the test is progressing as expected. The EBC then flows through the microfluidic material over the detection well and detection area of the biosensor and then makes contact with a second fluid detector where the change in conductivity in the gap between the adjacent conductors indicates that EBC has successfully flowed over the biosensor and a valid test in being performed.

The present invention provides a usability workflow for a self-contained mask-based diagnostic (MBD) that simplifies the testing process. The user only needs to put the mask on and breathe normally, and exhaled breath vapor is converted into a liquid biosample, EBC. The EBC is collected in a pool and transferred using microfluidic materials to flow over the detection area of the g-FET biosensor. A first liquid detection circuit detects when the EBC begins flowing and starts a 10 minute countdown. A spoken message is played through a speaker to inform the user that the test is in progress and to continue breathing normally for another 10 minutes. Each minute, a countdown reminder is provided as subsequent spoken messages. If the EBC flow doesn't begin stops for any reason before an adequate quantity has been collected during the test period, a spoken message tells the user that the test is not valid, and they should dispose of the mask and try again with a new mask. After the 10 minute test period ends, a spoken message informs the user if they are Covid-19 positive or negative, and at the end of testing, the user is instructed to remove the mask and seal it in the provided bag, dispose of the mask and bag in a trash can.

In contrast to existing OTC Covid-19 tests, the MBD provides optimal specificity and selectivity criteria. Biosample collection is not tedious or uncomfortable, and the MBD requires no complex sequence of actions by the user. The testing components are easy to handle, even for individuals with age, visual or motor skills related disabilities. Distinctive technical characteristics of the MBD are the use of an EBC biosample and a g-FET biosensor, which exploit the clean, low ionic EBC sample. The MBD requires the user to do nothing more than put on the mask and breathe, which is a stark contrast to the best OTC test currently available.

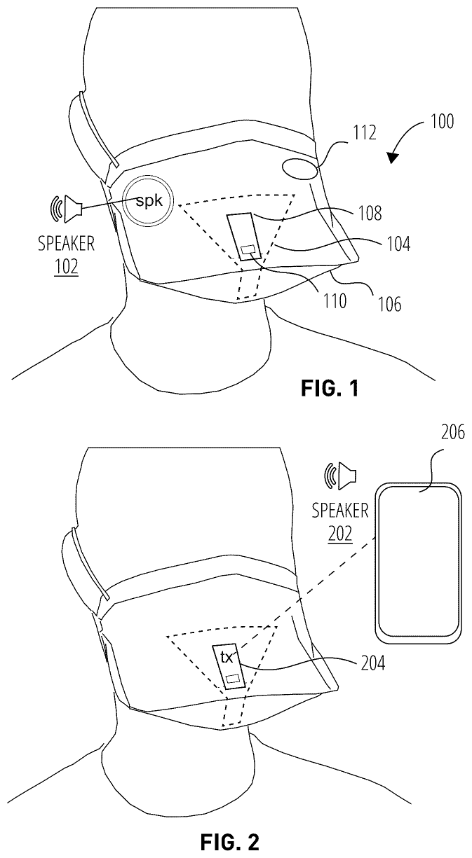

FIG. 6 is a flow chart of an embodiment where a user just has to press a push button on the mask (e.g., the MBD shown in FIG. 1) or press an onscreen or physical push button or give a voice command using a smartphone (e.g., the MBD shown in FIG. 2).

The user indicates it is time to start the test, by pressing a start button or voice command, depending on the user-interface (step one). Alternatively or additionally, the start of the test can be determined automatically using a fluid detector that detects when EBC has begun to flow (indicating the mask is being worn and EBC has started to flow). A start message is played, for example, audibly indicating to the user to “put the mask on and breath normally” (step two). If the expected test duration is 10 minutes (as an example), then a countdown timer is triggered at the start of the test (step three). The flow of the EBC is continuous checked using the fluid of liquid detectors. For example, if a first liquid detector (LD1) is located at the start of the flow of EBC through the fluid conductor, when the EBC is detect, a message can be played indicating to the user that the test is proceeding as expected (step five). If the countdown timer reaches a first minute interval since the start of the test, a message can be played encouraging the user to continue breathing (step seven). Similar countdown messages can be played at each minute interval to keep the user encouraged to continue to just breathe normally.

When a point in the test is reached, such as at minute 5 since the start of the test (step eight), if the EBC has not yet been detected (step nine) the test is declared invalid and the user instructed to try again with another MBD (step ten). If the EBC was detected, then the progress message “keep breathing for 5 more minutes” can be played (step eleven). The countdown timer continues on, playing an encouraging progress message at the end of each minute interval until the countdown timer reaches 0 minutes left (CD=T-0) (step twelve). At the end of the test duration, if the EBC biosample has not been detected (step thirteen) during the test duration by the fluid detector (LD2) located in the flow path after the biosensor, then the test is declared invalid (step fourteen). If the EBC has been detected (step thirteen), then if the result is negative (step fifteen) a “test is negative” message is played (step sixteen). If the result is not negative (step fifteen) and the test if determined to be positive (step seventeen) than a “test is positive” message is played (step eighteen). If the test is not determined to be either negative (step fifteen) or positive (step seventeen), then the test is declared invalid and the “test is invalid” message is played (step fourteen).

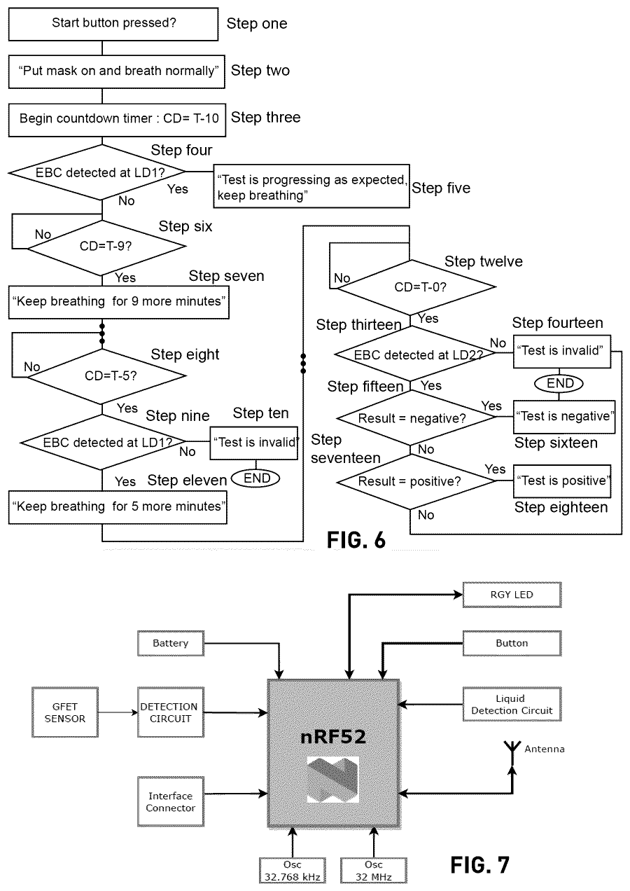

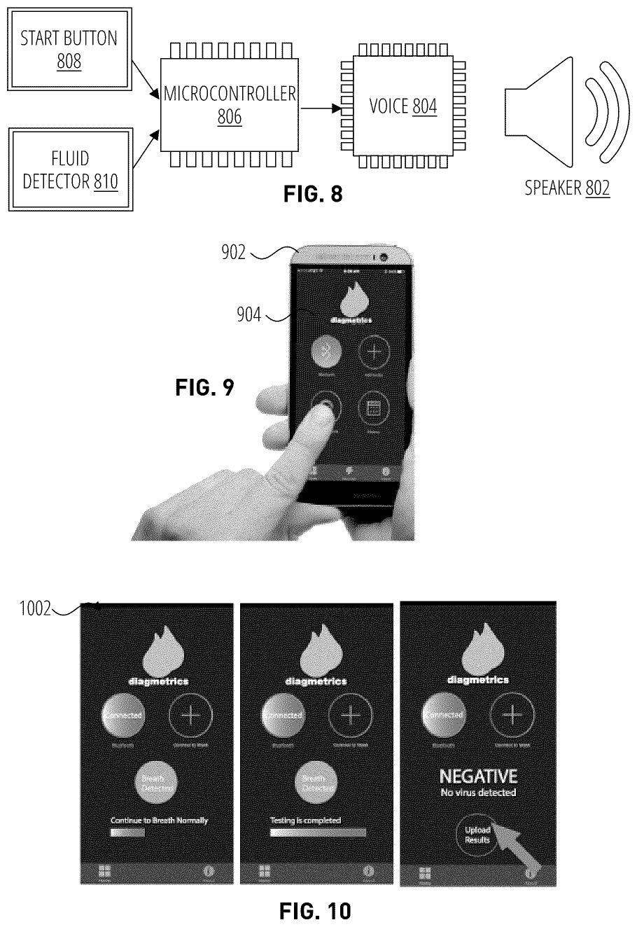

FIG. 7 is a block diagram of the electronic circuit of an EBC test unit. The EBC test unit includes a biosensor 202, a fluid detector 306, a battery 402, analysis electronics 302, communications electronics 306, and one or more test result/progress indicating LEDs 606 may also be provided (for example, to provide a self-contained testing system that can be used by a hearing impaired individual). The analysis electronics detect the target molecule dependent on an electrical signal received from the electronic biosensor. The various components can be incorporated as elements on the printed circuit board or as components of a system-on-chip package which may also include the biosensor. The communications electronics communicates the detection of the target molecule to a user through an indicator, such as a light emitting diode or smartphone, computer or tablet display. The communications electronics can communicate the detection of the target molecule wirelessly through an optical or radio frequency (RF) signal, for example, Bluetooth communication to a smart phone or network relay device.

The fluid detector may comprise a pair of adjacent electrical conductors in a fluid detection region of the flow path. A change in conductivity between the pair is detected when the EBC sample flows in the fluid detection region. The fluid conductor comprises a micro-fluidics material that is selected to reduce the adhesion of the target molecule to limit target molecules from being removed from the portion of the EBC sample that flows in the flow path while the target molecule is carried along with the EBC sample to the electronic biosensor.

The spoken word messages can include progress messages audibly provided during the testing process, including at least one of a message to alert the user that the test is in progress and to keep breathing normally triggered when said at least one fluid detector detects the start of fluid conduction through the fluid conductor.

A valid test is determined depending on if the fluid detectors detects the fluid conduction of the liquid sample past the biosensor. The spoken word messages can include countdown messages audibly provided during the testing process to provide a countdown of time remaining for the user to continue to breathe into the face mask. The spoken word messages can also include a test result message audibly provided during after the testing process is complete.

A self-contained version of the MBD system with accessibility features includes a speaker 802, a voice chip 804, a microcontroller 806, a start button 808, and a fluid detector 810. The mask-based may also include where the electronics includes a self-contained electronic audio circuit, the circuit include a spoken word generator that uses at least one of synthesized and recorded spoken messages played through at least one of a speaker and a headphonejack.

The smartphone user interface (UI) version of the MBD with accessibility features has wireless Bluetooth communications to transmit the test results relayed through a smartphone via the internet to trusted receivers (HCPs) and for aggregation for public good (public health authorities, contract tracing) and population studies (e.g., AI/ML Big Data analysis). The MBD ensures patient privacy while still enabling important health data to be transmitted for patient treatment (remote patient monitoring) and for aggregation by public health authorities. The test report can be sent to a trusted receiver, such as a patient's healthcare provider or insurance company, with encrypted patient identifying information. If the report is not for a trusted receiver but instead is for contact tracing or a population study, then only the required data is transmitted to a known Contact Tracing System, and the minimum patient identifying information is stored along with the test report in compliance with privacy regulations and/or agreements.

A smartphone UI version of the MBD system includes an APP 1002 running on the smartphone. The APP enables the smartphone to act as a convenient user-interface. The MBD may also include a wireless transmitter to transmit a wireless signal to control at least one of a smartphone, smart speaker, computer or accessibility device to generate the audible spoken word messages. FIG. 9 shows screens of a smartphone APP providing a graphical user interface for conveying the steps for conducting a test using a mask-based diagnostic device and receiving information indicating the test results.

The EBC testing unit described herein can be configured with bluetooth communication processor and/or microcontroller. The electronic biosensor produces a direct-to-electrical signal output. That is, there is no visual or optical test result, since the biosensor detects a target molecule through a mechanism that results directly into a change of electrical output detectable at the connection leads to the sensor constituents (e.g., source, drain, gate leads). This direct-to-electrical signal is particularly suited for low cost digital communication and signal processing. The signal from the biosensor can be sent as a raw value, which is then analyzed, for example, using the processing power of a cellphone or network server, or the signal from the biosensor can be processed by a microcontroller or processor provided in direct electrical communication with the biosensor, such as contained on the EBC testing unit PCB.

The processing of the signal can be split among devices, with wired or wireless signal transmission among the devices. For example, a smartphone and APP can be used as shown to indicate instructions for use and a final test result to the user or healthcare provider administering the test to a patient. The mask-based diagnostic device is particularly useful as an at-home or point-of-care test, where a test subject only has to open the APP on their smartphone, put on the mask and breathe. Such a simple test system can change the course of disease outbreaks, such as viral pandemics, and be used to proactively monitor an at-risk patient for many diseases and health conditions that are detectable from biomarkers contained in blood, lungs, airways and exhaled breath of the test subject. Also, the mask-based diagnostic device is particularly suitable for aggregating data obtained from large populations regionally located or scattered throughout the world by using existing network infrastructures, such as cellular networks and the Internet, to obtains copious data useful, for example, by Artificial Intelligence (AI) and/or Machine Learning (ML) algorithms.

The system comprises a collection pool 1102, a mask 1104, and an EBC collector 1106.

The MBD system comprises a fluid conductor 1202, an MBD 1204, an NFC 1206, a fluid conductor 1208, a biosensor 1210, and an EBA co-collector 1212. The fluid conductor can also be formed as a hydrophilic channel disposed on a hydrophobic field. For example, the condensation surface of the EBC Collector can be a thin Teflon or PTFE film (or other hydrophobic material). The Teflon film can have hydrophilic channels formed by selectively etching or treating the Teflon film through a shadow mask or, for example using a scanning laser, selectively ablating the surface of the Teflon film, so that higher surface energy channels are formed on the relatively lower surface energy field of the Teflon film surface. Additionally, or alternatively, the fluid conductor can be formed by patterning an electrospun or screen printed Super Absorbent Polymer (SAP) structure that provides a fluid conduction flow path so that the collected EBC is transported to the detection well of the biosensor. The SAP can be formulated and structured to selectively absorbs water and ions from the EBC sample and concentrate the target molecules in the portion of the EBC sample that flows over the biosensor.

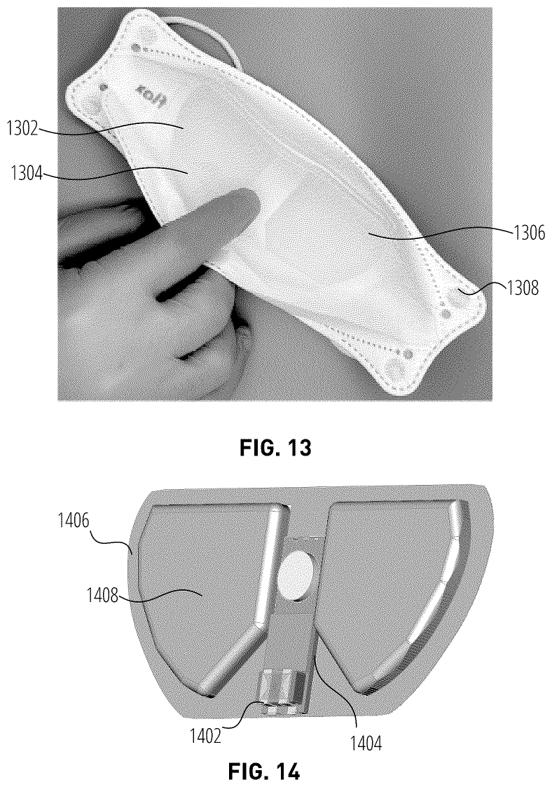

FIG. 13 shows a mask-based diagnostic device. The system comprises an EBC collector 1302, an MBD 100, and a thermal mass 1306. The MBD 100 defines, during use, a confined local environment. The local environment includes breath vapor exhaled from the lungs of a user. The EBC collector 1302 has a condensate forming surface 1304 that converts exhaled breath vapor into an EBC liquid sample. The EBC collector 1302 includes a thermal mass 1306 cooled before use so that during use the condensate forming surface is at use to a condensation forming temperature less than a confined environment temperature of the confined environment inside of the face mask.

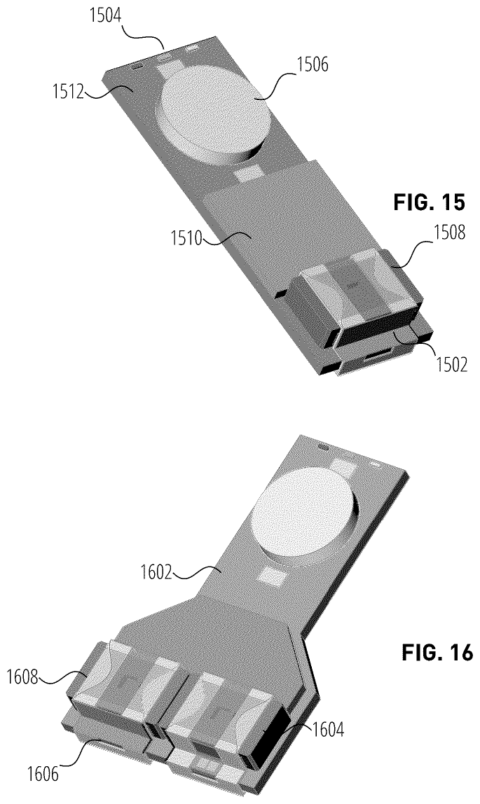

FIG. 14 illustrates an EBC collector with a centrally mounted EBC testing unit. The system comprises an EBC collector 1302, a condensate forming surface 1304, a biosensor 302, and an EBC testing unit 306. The EBC testing unit test the EBC sample for a target molecule, where the EBC sample contains water and the target molecule. The EBC testing unit includes a printed circuit board supporting a semiconductor packaged electronic biosensor in electrical communication with power, analysis electronics and communications electronics. A fluid conductor conducts the EBC sample to the electronic biosensor. A fluid detector onboard the printed circuit board detects at least one of a start of fluid conduction through the fluid conductor in a flow path before the electronic biosensor and an end of fluid conduction in the flow path after the electronic biosensor. The analysis electronics detects the target molecule dependent on an electrical signal received from the electronic biosensor. The communications electronics communicates the detection of the target molecule. The analysis electronics and the communications electronics, biosensor, printed circuit board, electronics, etc., can also be deployed in an different EBC testing unit format. For example, a lateral flow assay (LFA) testing unit can be utilized, with some of the EBC testing unit components changed or obviated, and other included. In the FIG. 15 illustrates the EBC testing unit showing a dissolvable surfactant film disposed in the fluid flow path before the sample flow reaches the electronic biosensor. This embodiment of the EBC testing unit includes a biosensor 302, a wick 304, a printed circuit board 312, a battery 402, an LED 408, and also a surfactant film 1502. The EBC fluid sample includes analytes or target biomarkers in a mostly-water fluid carrier. The dissolvable surfactant film is placed in the fluid flow path before the sample flow reaches the electronic biosensor so that, for example, a lipid viral shell of the SARS-CoV-2 virus can be breached through the chemical action of the surfactant on the lipids making up the viral shell. This causing lysing of the virus to release internal biomarkers, such as the virus N− protein, which can then be detected using a biosensor that is functionalized with capture molecules specific to binding to the N− protein.

The biosensor incorporated in the present invention is highly selective and sensitive, enabling accurate detection of very low viral counts in exhaled breath. The biosensor is functionalized with a capture molecule(s) that is specifically designed to target the biomarker(s) of interest. The testing platform has been designed as a flexible system that can accommodate multiple capture molecules, which allows for testing of multiple biomarkers. If a new variant emerges that is not detectable, the system can be easily modified to add a new capture molecule specifically designed for the new variant. This allows for the constant addition of capture molecules to the biosensor to detect and identify infections from new variants.

FIG. 16 illustrates a version of the EBC testing unit showing two parallel biosensors for simultaneously receiving the fluid sample at the detection area of each biosensor. In many use-cases it is advantageous to test for two or more different biomarkers in one tested sample. For example, a person infected with SARS-CoV-2 can be tested for both the S− or spike protein of the virus. These S− proteins are typically found on the outside of the virus particle and can be detected by a biosensor that is functionalized with a capture molecule designed to specifically bind to this target biomarker, the S− protein. In parallel, or sequentially, the EBC sample can be directed to flow to a second biosensor as well. In this case, prior to reaching the second biosensor the EBC sample can be treated with a lysing chemical (e.g., contained in a dissolvable surfactant film) to release a second detectable biomarker, such as the virus N− protein. The second biosensor is functionalized with the capture molecules that are designed specifically to bind to this second biomarker, the N− protein.

It may be advantageous to allow the EBC or liquid sample to flow over the detection area, allow flow volume and time to elapse so that the target molecules bind to the capture molecules, and then before taking a test reading of the electrical signal generated by the biosensor, rinse the detection area of the biosensor with a clean solvent, such as de-ionized water. The rinse will remove from the detection area at least some of the potentially test-confounding molecules and ions that may be present in the EBC, including surfactant, lysing materials and other non-target molecules and ions. The target molecules that bind with the immobilized capture molecules will be held at the detection area. The rinse can be done intermittent with the application to portions of the test sample to the detection area, so that confounding molecules can be rinsed away giving additional target molecules more opportunity bind with the immobilized capture molecules and change the electrical characteristics of the biosensor.

The system is also easily adaptable to detect and identify other infectious diseases, such as FluA/B. This is particularly useful during the flu season when a syndromic testing system can be provided that tests for all SARS variants, as well as FluA/B. The biosensor may be, for example, a g-FET biosensor functionalized with nanoCLAMP™ capture molecules. The use of multiple capture molecules in the testing platform ensures accurate and reliable results, and the ability to add new capture molecules as needed allows for continued testing of new variants and infectious diseases.

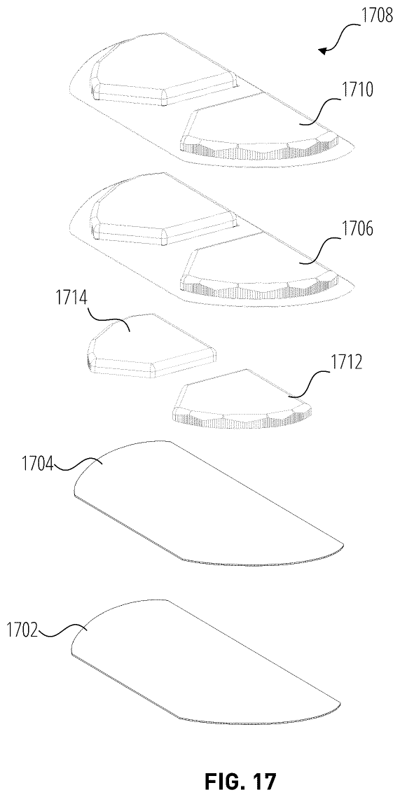

FIG. 17 is an exploded view showing the components of the EBC collector. The components of the EBC collector 1302 include the thermal mass 1306, a condensate forming surface 1304, an attachment member 1702, a support member 1704, and adhesive sheets 1706. The condensate forming surface can comprise a low surface energy material, such as Teflon sheet, to reduce the adhesion of the target molecule to limit target molecules from being removed from the collected EBC sample. The thermal mass is disposed between two sheets of adhesive, where one of the adhesive sheets can be thermo formed to provide a pocket for the thermal mass, and supports the low surface energy Teflon sheet comprising the condensate forming surface. The thermal mass can comprise a water and SAP polymer gel disposed between the sheets of adhesive. A collection pool can be provided for receiving the EBC, where the fluid conductor conducts the EBC from the conduction pool to the EBC testing unit. Another one of the adhesive sheets is also a support member 1704 and the components of the EBC collector are configured and dimensioned so that the EBC collector can be retrofit into a pre-existing face mask. The support member can be made from other materials, such as a plastic sheet or foil. This configuration allows for the creation of a mask-based diagnostic device using many of the available face masks from a large number of world-wide manufacturers. An adhesive attachment member 1702 can have a pressure sensitive adhesive side that is formulated to stick to the inside surface of the pre-existing face mask. The faces that come in contact with each other of either or both the support member and the attachment member can have surface properties that facilitate easy removal and replacement of the attachment member so that the EBC collector can be removed after use from a disposable face mask with the attachment member left on the inside surface of the face mask. The EBC collector can then be sanitized, a new attachment member added, and recycled into another mask-based diagnostic device.

An embodiment designed for scalable roll-to-roll manufacturing includes mostly sheet materials and a lost cost, safe and highly effective thermal mass. In prototype form, the sheet materials that form the EBC condensation surface, thermal mass and support member are cut to 6″×2.5″. A heat press and die or thermal vacuum forming process is used to form a thermal mass pocket in a lamination of 0.005″ Teflon sheet (condensation surface) and double sided 3M 96042 adhesive. The 3M 96042 adhesive has a low surface energy (LSE) adhesive coated on both sides of a polyester liner, and is an example of the adhesives available that can be used for this embodiment. About 2 ml of a water/SAP gel (thermal mass) is deposited into the thermal mass pocket. This water-based thermal mass takes advantage of the high specific heat capacity of water making the chilled water-based thermal mass well suited for this application of cooling the Teflon condensation surface during a period of time needed to collect an adequate sample of EBC.

The 3M 96042 adhesive is a low surface energy adhesive that is specifically designed to stick to difficult to adhered to surface, and sticks well to the Teflon sheet and to itself, so two sheets of 3M 96042 can be used to form a water-tight enclosure for the water/SAP gel thermal mass. A second sheet of the 3M 95042 adhesive forms the support member and seals the water/SAP in the thermal mass pocket. Since the 3M 95042 remains sticky even when covered with water, a 0.003″ polyester sheet or tissue paper spacer is cut to fit within the pocket to prevent the two adhesives from sticking to each other at the location thermal mass. The collection pool is made from the same Teflon sheet material, adhered in place with the 3M 96042 adhesive to form a water-tight pool. This construction results in a very robust EBC collector that can withstand multiple sterilizations and re-uses, and is water tight and has a thermal mass pocket that is difficult to pop or tear open.

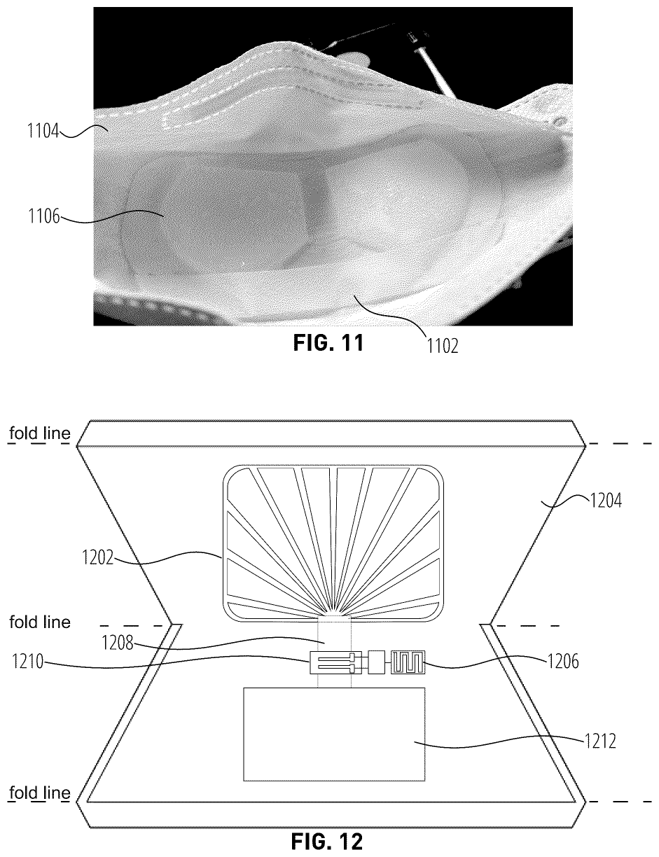

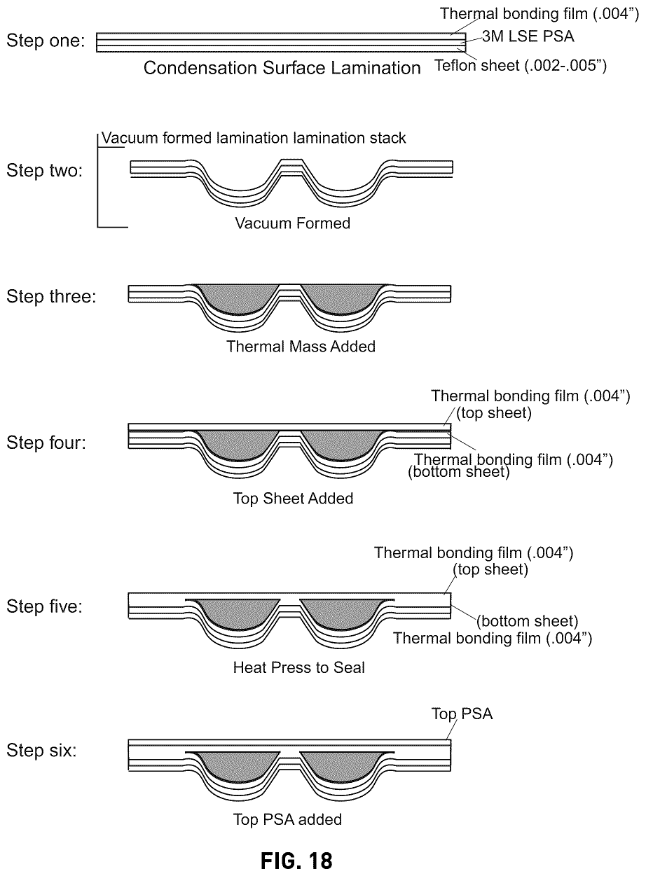

FIG. 18 illustrates another exemplary manufacturing process for forming the EBC Collector. A condensation surface lamination is formed that has a PTFE front face adhered to a thermal bonding film by a pressure sensitive adhesive (Step one). A thermal vacuum formation process (Step two) forms the thermal mass receiving pockets in the lamination. The thermal mass is added (Step three). A top bonding film is provided (Step four) and bonded to the bottom thermal bonding film (Step five) to seal the thermal mass within the two pockets. A top PSA can be added (Step six) that is used to adhere the EBC collector to the inside of a pre-existing face mask (e.g, shown in FIG. 11) or to a foldable mask substrate (e.g., shown in FIG. 12).

The thermal mass can be plain water, water plus SAP, or an endothermic two part ingredient where each ingredient is kept separate until mixed and an endothermic reaction actively cools the condensation surface during use of the MBD. For example, water can be provided in one pocket and urea crystal provided in the other. The space between the two pockets is bonded with enough bond strength to keep the ingredients separated until pressure is applied by a user to break the bond between the two pockets and allow the ingredients to mix together and endothermically cool the condensation surface.

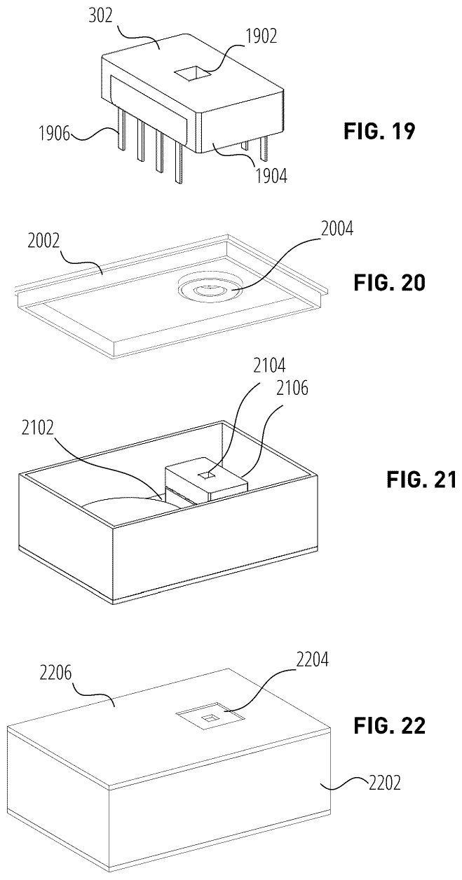

FIG. 19 is an isolated view of a semiconductor DIP packaged biosensor for incorporation in a printed circuit board. The semiconductor DIP package comprises a biosensor 302, a detection well 1902, an encapsulant 1904, and pins 1906.

The functioning of the g-FET biosensor requires access to a portion of the top surface of the semiconductor device. At the top surface the detection area is provided with the charge transport layer of graphene, and on the graphene layer capture molecules are immobilized that are designed to bind to the target biomarker molecules and thereby cause a detectable change in the electrical output of the g-FET. However, this necessary feature of leaving a portion of the top surface of the semiconductor device exposed is unusual and requires a different semiconductor package than is usually available for packaged semiconductor electronic devices. As a result, g-FET biosensors are typically sold as bare die components with connecting pads for connecting the g-FET features (e.g., source, drain and gate) to typical research lab equipment, such as a probe station. To make a g-FET biosensor scalable for mass production, a solution is needed to package the g-FET bare die so that it can be conveniently connected with a printed circuit board. In accordance with another non-limiting embodiment, a semiconductor DIP packaged biosensor is provided where the conventional Double In-line package, DIP, configuration is used to make the biosensor easy to handle and easy to place into a printed circuit board. There are other semiconductor package types that can be used in accordance with the embodiment shown, including, for example, SOP/SOIC/SO (Small Outline Package), QFP (Quad Flat Package), QFN/LCC (Quad Flat Non-leaded Package, BGA (Ball Grid Array Package) and CSP (Chip Scale Package). In all cases, access to a portion of the top surface of the semiconductor g-FET device is provided through a window or well built into a top portion of the packaging encapsulating material, which could be formed in place with the window or well, or provided as a pre-formed lid having the window or well. This detection well in the semiconductor packaged electronic devices obtains a configuration of the electronic biosensor that can be easily handled by existing electronic circuit production equipment at scale.

As described herein, embodiment of the mask-based diagnostic device can include an EBC testing unit including a PCB with packaged electronic biosensor that are mounted directly onto the EBC collector. This allows a self-contained system that is well suited for massive deployment for at-home testing and other-the-counter sales. However, the collection of the EBC sample and the testing of the collected sample can be done remote from each other. For example, the EBC collector can be used to obtain an EBC sample, and then the sample sealed within the collection pool as shown herein. The accumulated EBC sample can also be removed from the collection pool using a pipette or dropper, placed in a vial and sealed. In these cases, the EBC sample can be transported to a remote lab for analysis. As another alternative, the collected EBC sample can be obtained immediately after the mask-based diagnostic device is used, and then the sample placed into the well of a table top EBC testing unit.

FIG. 20 illustrates the lid 2002 with an o-ring 2004 for use in a table top EBC testing unit.

FIG. 21 illustrates the housing in which sits an EBC testing unit PCB comprising a biosensor 302, a detection well 1902, and a socket 2102.

FIG. 22 illustrates an assembled table top EBC testing unit showing a testing well for receiving a liquid sample. The table top EBC testing unit is housed in a housing 2202, and includes a lid 2002 with a testing well 2204. In this case, the test subject does not have to wait for the EBC sample to be transported to a remote lab, wait for the lab to perform the analysis of the EBC, and then wait even longer to get the test results either directly from the lab or relayed through a health care provider. The table top EBC testing unit can be used at the same location where the EBC sample is obtained, and is suitable for clinical or at-home use.

Also, the table top EBC testing unit can be used for detecting biomarkers and target molecules in other fluids. For example, blood, urine, saliva, sweat, interstitial fluid, tears, mucus, gastrointestinal lavage, or other bodily fluid. Also, the small size and portability, along with the wireless smartphone APP-based user interface and network relay communications capabilities as described herein can be used for environmental testing of waste water, potable water, HVAC condensate and room-scale environmental and bio-sensing.

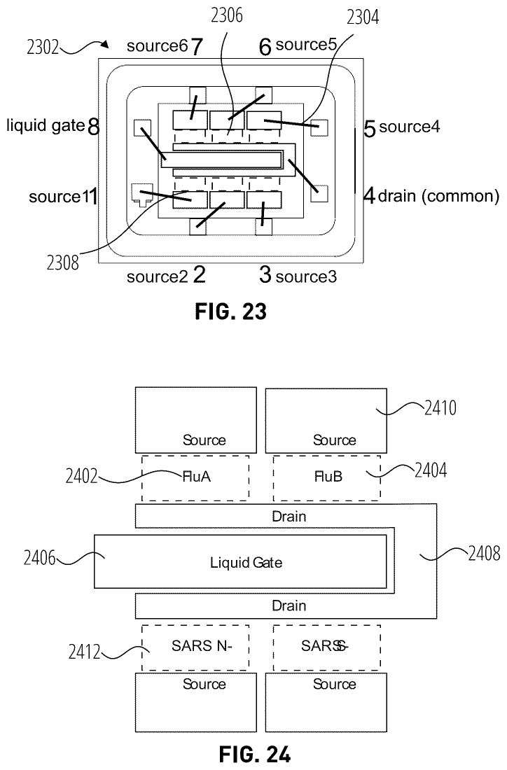

FIG. 23 illustrates a single bare die wire bonded for semiconductor packaging and having multiple and differently functionalized g-FET biosensors. The charge transfer layer 2306 of each g-FET 2308 on the bare die semiconductor device is functionalized with a different set of capture molecules. A DIP package 2302 is constructed using conventional semiconductor electronic circuit device materials and processes including wire bonds 2304 connecting the electrodes of the g-FET biosensor features (sources, drains, liquid gate) to respective pins of the DIP package. An encapsulation process encases the bare die and leaves an opening so that the detection areas of the g-FETs are accessible by the sample being tested.

FIG. 24 illustrates a bare die biosensor having g-FET devices functionalized for FluA/FluB/SARS virus testing. In this case, each g-FET on the bare die semiconductor device is functionalized for detecting if the test sample contains a biomarker of FluA, FluB or SARS. In this example, the SARS biomarkers include both the SARS N− protein and S− protein. Each charge transfer layer 2306 of the different g-FETs has a different type of capture molecule (e.g., capture molecule1 2402 for detecting FluA biomarker). The capture molecules are immobilized at the detection area 2404 of each corresponding g-FET. A liquid gate electrode 2406, a drain electrode 2408, and a source electrode 2410 provide electrical conduction to the semiconductor features that form the different biosensors ganged on semiconductor bare die, where one or more of these biosensors can be functionalized at the wafer level.

The present invention is designed as a modular system of subassemblies, with each module being completed and tested to ensure their functionality. The packaged biosensor can have multiple g-FET sensors on a single bare die, and each sensor can be individually functionalized with a different capture molecule. This allows for the creation of a syndromic biosensor that can test for multiple biomarkers of the same disease (e.g., S and N proteins and even RNA of SARS-CoV-2 virus) and/or different diseases (e.g., SARS, Flu-A, Flu-B).

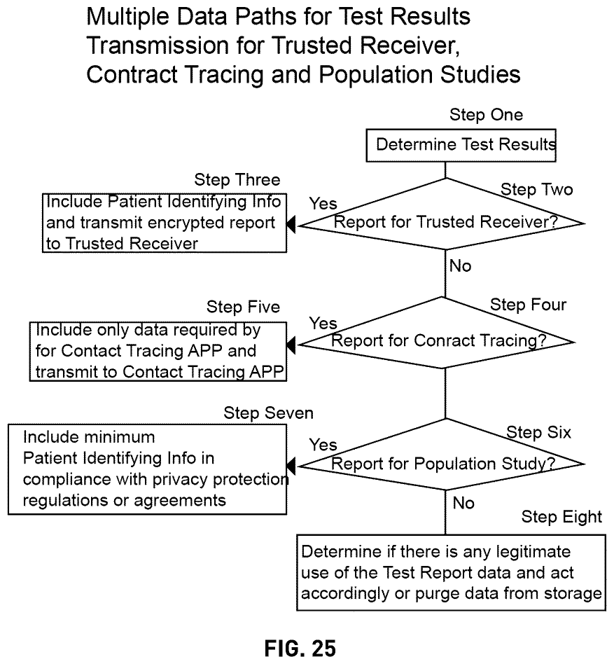

The flow chart shown in FIG. 25 illustrates a use of the MBD as a remote patient monitoring device (RPM) that automatically configures a transmitted data stream so that the appropriate level of patient identifying data is transmitted depending on the transmission path and the receiving entity. For example, the version of the MBD with accessibility features that uses a smartphone or other external device as the user interface can also include choices selectable by the user, caregiver or test administrator so that once a test results is obtained that result can be transmitted to the Dr or HCP of the patient (user) including patient identifying data and two-step verification, password protection, etc.

A wireless receiver receives the test result signal, determines a test result from the test result signal, and relays to a remotely located network server the test result and test subject identifying information. The wireless receiver includes a processor including computer code for determining a type of test report to be sent to the remotely located network server. The type of test report relayed to the remotely located network server is determined by the processor dependent on at least two of three types predetermined types of test reports and corresponding types of uses for the test report including trusted receive use, contact tracing use, and population study use.

If the test report is determined to be sent for the trusted receiver use, an encrypted trusted receiver test report is transmitted to the remote server for use by the trusted receiver and includes test subject identifying information including patient identifying data for identifying to the trusted receiver the presence of the detected biomarker in the exhaled breath of the test subject. If the test report is determined to be sent for the contact tracing use, a contact tracing test report is transmitted to the remote server for use by a contract tracing algorithm and includes test subject identifying information including only contact tracing data required for performing contact tracing by software running on the remote server to determine individuals who have come in contact with the test subject. If the test report is determined to be sent for the population study use, a population study test report is transmitted to the remote server for use by a population study algorithm for Blockchain and AI database collection, access and analysis including minimum test subject identifying information in compliance with privacy protection regulations and agreements.

The flowchart shows the logic flow for data acquisition and transmission for trusted receiver and contract tracing uses. The performance of the work flow can be done at the testing system, network node, Smartphone, or combination of components located or associated with the test subject through the data receivers(s) (Dr., HCP, Government or NGO, etc.) or final storage location(s) of the acquired data. The acquired data can include patient or subject identifying information ranging from name, GPS location, list of known contacts, prior medical history, demographics, etc.

The digital testing system architecture, manufacturing methods, and applications, described herein, or others, can be used for capturing biometric data from the exhaled breath of a test subject or patient. Biometric data can be captured and transmitted continuously or at selected times with data access provided directly to a care-provider, enabling early diagnosis and ongoing monitoring, and to a researcher to gain valuable insights and assistance through AI analysis. This data detection is direct from the exhaled breath and can be provided through a wireless connection for Blockchain and AI database collection, access and analysis.

Referring the flow chart in FIG. 25, a Test Result or Report is determined or is received (step one) (e.g., from a Smartphone transmission from the patient or test subject). If the report is intended to be sent to a trusted receiver (step two), such as a patient's healthcare provider or insurance company, then an encrypted report can be generated (step three) and transmitted to the trusted receiver that includes patient identifying information. If the report is not for a trusted receiver (step two) but instead is to be used for contact tracing (step four), then only the data required for Contact Tracing is transmitted to a Contact Tracing APP (step five). The Contact Tracing APP may be, for example, a system provided for identifying and notifying people who have come in contact with the test subject or patient within a given time prior or since testing positive or maybe for one or more target biomarkers. If the report is not for a trusted receiver (step two) or for contact tracing (step four) but instead is to be used for a population study (step six), then only the minimum patient identifying information in compliance with privacy regulations and/or agreements is transmitted and/or stored along with the received test report (step seven). If the report is not for a trusted receiver, contact tracing or population study (step six) then it is determined if there is any legitimate use of the test report data and an action is taken accordingly or the automatically data is purged from storage.

The present invention can be used as a remote patient monitoring (RPM) system, which allows healthcare providers to monitor patients outside of a clinical setting. RPM is likely to become an essential tool to proactively assess patients' health and enable timely intervention before an adverse event occurs. We are nearing the minimum viable product (MVP) for a diagnostic platform that includes the electronics for Bluetooth communication and encryption of data obtained as a direct-to-electrical signal generated by the g-FET biosensor. Encrypted test results can be relayed from a smartphone through the internet to trusted receivers and public authorities. The test results can be automatically wirelessly transmitted and displayed in minutes from putting the mask on and breathing normally. The MBD offers an ease and frequency of testing that is not practical with any other available Covid-19 testing system.

The MBD can be reconfigured quickly for testing other potential pandemic diseases, such as tuberculosis, which is increasingly becoming drug resistant and remains one of the world's deadliest infectious diseases. While RT-PCR remains the gold standard, our mask-based diagnostic platform allows faster and more cost-effective testing that can be implemented on a larger scale. The modular design of the MBD allows for flexibility and adaptability to meet the ever-changing needs of pandemic response and patient care.

Prototypes of the MBD with accessibility features have been constructed demonstrating how a simple “put the mask on and breathe” diagnostic device can be particularly useful for members of a targeted community, such as those with low vision, blindness, cognitive and/or motor impairments, young age, or elderly with age related disabilities and/or differing levels of technology savvy and/or access to external devices, such as a smartphone. Below is a program written in Arduino that controls the operation of an MP3 player module and other electronic components connected to an Arduino board. This software and electronics have been demonstrated on the version of the MBD with accessibility features where the spoken word messages and the speaker are provided on the mask. A similar code can be used for the version of the MBD with accessibility features where the user interface is a smartphone, smart speaker, accessibility device or the like.

The program initializes connections from the microcontroller to the MP3 player module and other components, including a start button, two switches, and two LEDs. When the start button is pressed, the program begins playing a set of spoken instructions and waits for a mask-is-cold message to be played. Once this message is played, the program starts a countdown timer that plays a series of spoken countdown messages at 60-second intervals. The program also checks for the closing of two liquid detection circuits, indicating the presence of exhaled breath.

When the countdown timer finishes, the program determines the test result based on the analysis of the EBC and validity of the test can depend on whether one of the liquid detection circuits is closed or not. A positive, negative or invalid test result message is played and the test ends.

There is also a reset function that sets all variables to their initial values to prepare for the next test.

| | |
|---|---|
| 1. | #include <SoftwareSerial.h> |
| 2. | #include “DFRobotDFPlayerMini.h” |
| 3. | SoftwareSerial ss(6, 7); |
| 4. | DFRobotDFPlayerMini myDFPlayer; |
| 5. | bool countdownStarted = false; |
| 6. | bool resetPressed = false; |
| 7. | bool LD1switchClosed = false; |
| 8. | bool LD2switchClosed = false; |
| 9. | bool instructionsPlayed = false; |
| 10. | bool maskIsColdMessagePlayed = false; |
| 11. | bool countdownFinished = false; |
| 12. | bool testResultPlayed = false; |
| 13. | unsigned long currentMillis; |
| 14. | unsigned long startMillis; |
| 15. | |
| 16. | const int startButtonPin = 0; |
| 17. | //const int resetButtonPin = 1; |
| 18. | const int LD1switchPin = 1; |
| 19. | const int LD2switchPin = 2; |
| 20. | const int actPin = 8; |
| 21. | const int instructionsMessage = 1; |
| 22. | const int startMaskIsCold = 2; |
| 23. | const int startMaskIsNotCold = 3; |
| 24. | const int m9min = 4; |
| 25. | const int m8min = 5; |
| 26. | const int m7min = 6; |
| 27. | const int m6min = 7; |
| 28. | const int m5min = 8; |
| 29. | const int m4min = 9; |
| 30. | const int m3min = 10; |
| 31. | const int m2min = 11; |
| 32. | const int m1min = 12; |
| 33. | const int invalid = 13; |
| 34. | const int negative = 14; |
| 35. | const int positive = 15; |
| 36. | const int EBCdetected = 16; |
| 37. | |
| 38. | void setup( ) { |
| 39. | Serial.begin(115200); |
| 40. | Serial.println(“DFPlayer Mini MP3 Player”); |
| 41. | ss.begin(9600); |
| 42. | |
| 43. | if (!myDFPlayer.begin(ss)) { |
| 44. | Serial.println(F(“Unable to begin:”)); |
| 45. | Serial.println(F(“1.Please recheck the connection!”)); |
| 46. | Serial.println(F(“2.Please insert the SD card!)); |
| 47. | while (true); |
| 48. | } |
| 49. | Serial.println(F(“DFPlayer Mini online.”)); |
| 50. | |
| 51. | myDFPlayer.volume(30); |
| 52. | |
| 53. | digitalWrite(LD1switchPin, HIGH); |
| 54. | digitalWrite(LD2switchPin, HIGH); |
| 55. | |
| 56. | pinMode(startButtonPin, INPUT_PULLUP); |
| 57. | //pinMode(resetButtonPin, INPUT_PULLUP); |
| 58. | pinMode(LD1switchPin, INPUT_PULLUP); |
| 59. | pinMode(LD2switchPin, INPUT_PULLUP); |
| 60. | pinMode(actPin, INPUT_PULLUP); |
| 61. | } |
| 62. | |
| 63. | void loop( ) { |
| 64. | if (digitalRead(startButtonPin) == LOW) { |
| 65. | if (!instructionsPlayed) { |
| 66. | instructionsPlayed = true; |
| 67. | Serial.println(“Start Switch Pressed”); |
| 68. | myDFPlayer.play(instructionsMessage); |
| 69. | Serial.println(“General Instructions message played”); |
| 70. | // Add a delay here for the general instructions message to play |
| 71. | delay(25000); |
| 72. | } else if (instructionsPlayed && !maskIsColdMessagePlayed) { |
| 73. | myDFPlayer.play(startMaskIsCold); |
| 74. | Serial.println(“mask is cold message played”); |
| 75. | maskIsColdMessagePlayed = true; |
| 76. | delay(20000); |
| 77. | // Start the countdown timer |
| 78. | countdownStarted = true; |
| 79. | startMillis = millis( ); |
| 80. | countDownTimer( ); |
| 81. | } else if (maskIsColdMessagePlayed && countdownFinished) { |
| 82. | playTestResult( ); |
| 83. | } |
| 84. | } |
| 85. | } |
| 86. | |
| 87. | void countDownTimer( ) { |
| 88. | int firstFileNumber = 4; |
| 89. | int i = 0; |
| 90. | const int numMessages = 9; |
| 91. | const int messageInterval = 60000; |
| 92. | |
| 93. | const String countdownMessages[numMessages] = { |
| 94. | “You are doing great. Keep breathing into the mask for another 9 minutes”, |
| 95. | “Great job. Keep breathing for more 8 minutes”, |
| 96. | “You are at minute 7, keep breathing normally”, |
| 97. | “Six more minutes to go”, |
| 98. | “You are halfway there, just five minutes to go”, |
| 99. | “Just four more minutes”, |
| 100. | “Three minutes left”, |
| 101. | “Two more minutes to go”, |
| 102. | “One more minute, then listen for your test results”, |
| 103. | }; |
| 104. | |
| 105. | while (i < numMessages) { |
| 106. | |
| 107. | currentMillis = millis( ); |
| 108. | |
| 109. | // Check if LD1 switch is pressed and the actPin is high |
| 110. | if (digitalRead(LD1switchPin) == HIGH && digitalRead(actPin) == HIGH) { |
| 111. | if (!LD1switchClosed) { |
| 112. | Serial.println(“LD1 detects EBC”); |
| 113. | myDFPlayer.play(EBCdetected); |
| 114. | Serial.println(“exhaled breath has been detected, continue to breath |

normally”);

| | |
|---|---|
| 115. | digitalWrite (LEDgreen, HIGH); |
| 116. | LD1switchClosed = true; |
| 117. | } |
| 118. | } |
| 119. | |
| 120. | // Check if LD2 switch is pressed |
| 121. | if (digitalRead(LD2switchPin) == HIGH) { |
| 122. | if (!LD2switchClosed) { |
| 123. | Serial.println(“LD2 detects EBC”); |
| 124. | digitalWrite (LEDblue, HIGH); |
| 125. | LD2switchClosed = true; |
| 126. | } |
| 127. | } |
| 128. | |
| 129. | // Play the countdown messages |
| 130. | if (currentMillis − startMillis >= (i * messageInterval) && currentMillis − |

startMillis < (i + 1) * messageInterval) {

| | |
|---|---|
| 131. | if (digitalRead(actPin) == HIGH) { |
| 132. | Serial.print (“countdown message ”); |
| 133. | Serial.print (i + 1); |
| 134. | Serial.println(“ played”); |
| 135. | myDFPlayer.play(firstFileNumber + i); |
| 136. | Serial.println(countdownMessages[i]); |
| 137. | i++; |
| 138. | } |
| 139. | |
| 140. | // Play the test results |
| 141. | if (i >= numMessages) { |
| 142. | delay (10000); //to allow time for 1 min to go message to play |
| 143. | countdownFinished = true; |
| 144. | if (LD2switchClosed) { |
| 145. | myDFPlayer.play(positive); //this program is not configured for actual |

EBC analysis, so we just play the positive test message for now

| | |
|---|---|
| 146. | Serial.println(“You tested positive for Covid-19”); |
| 147. | } |
| 148. | else if (!LD2switchClosed) { |
| 149. | myDFPlayer.play(invalid); |
| 150. | Serial.println(“Your test is invalid”); |
| 151. | } |
| 152. | return; |
| 153. | break; |
| 154. | } |
| 155. | } |
| 156. | } |
| 157. | } |
| 158. | |
| 159. | void playTestResult( ) { |
| 160. | if (LD2switchClosed) { |
| 161. | myDFPlayer.play(positive); |
| 162. | Serial.println(“Positive test result played”); |
| 163. | } else if (!LD2switchClosed) { |
| 164. | myDFPlayer.play(invalid); |
| 165. | Serial.println(“Invalid test result played”); |
| 166. | } |
| 167. | } |
| 168. | |
| 169. | void reset( ) { |
| 170. | countdownStarted = false; |
| 171. | resetPressed = false; |
| 172. | LD1switchClosed = false; |
| 173. | LD2switchClosed = false; |
| 174. | instructionsPlayed = false; |
| 175. | maskIsColdMessagePlayed = false; |
| 176. | countdownFinished = false; |
| 177. | testResultPlayed = false; |
| 178. | return; |
| 179. | } |

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

What is claimed is:

1. A mask-based diagnostic (MBD) system for providing test results using an exhaled breath condensate (EBC) biosample in less than 10 minutes, comprising: a mask having an EBC collector for converting exhaled breath vapor into a collected liquid biosample; a biosensor for generating an output signal dependent on the detection of a target biomarker in the liquid biosample; and electronics for determining a test result from the output signal and controlling the generation of audible spoken word messages for providing automatic readout of at least one of test instructions, test progress and the test results.

2. The mask-based diagnostic device of claim 1, wherein the electronics and biosensor are components of an EBC testing unit for testing the liquid sample for the target biomarker, where the EBC sample contains water and the target molecule, the EBC testing unit comprising the electronics formed on a printed circuit board supporting the biosensor provided as a semiconductor packaged electronic biosensor in electrical communication with power, analysis and communications electronics; and further comprising, a fluid conductor for conducting the EBC sample to the electronic biosensor.

3. The mask-based diagnostic device of claim 1, where the electronics provided on the printed circuit board have at least one fluid detector for detecting at least one of a start of fluid conduction through the fluid conductor in a flow path before the electronic biosensor and an end of fluid conduction in the flow path after the electronic biosensor.

4. The mask-based diagnostic device of claim 3, where the fluid detector comprises a pair of adjacent electrical conductors in a fluid detection region of the flow path where a change in conductivity between the pair is detected when the EBC sample flows in the fluid detection region.

5. The mask-based diagnostic device of claim 3, wherein the spoken word messages include progress messages audibly provided during the testing process, including at least one of a message to alert the user that the test is in progress and to keep breathing normally triggered when said at least one fluid detector detects the start of fluid conduction through the fluid conductor.

6. The mask-based diagnostic device of claim 3, wherein determines if a valid test has been obtained dependent on if said at least one fluid detector detects the fluid conduction of the liquid sample past the biosensor.

7. The mask-based diagnostic device of claim 1, wherein the spoken word messages include countdown messages audibly provided during the testing process to provide a countdown of time remaining for the user to continue to breathe into the face mask.

8. The mask-based diagnostic device of claim 1, wherein the spoken word messages include a test result message audibly provided during after the testing process is complete.

9. The mask-based diagnostic device of claim 1, wherein an inside of the face mask defines during use a confined local environment, where the local environment includes breath vapor exhaled from lungs of a user, and where the EBC is collected by an EBC collector having a condensate forming surface for converting the exhaled breath vapor into the liquid sample, the EBC collector including a thermal mass cooled at least one of before or during use so that during use the condensate forming surface is at a condensation forming temperature less than a confined environment temperature of the confined environment inside of the face mask.

10. The mask-based of claim 1, wherein the electronics includes a self-contained electronic audio circuit, the circuit include a spoken word generator that uses at least one of synthesized and recorded spoken messages played through at least one of a speaker and a headphone jack.

11. The mask-based of claim 1, wherein the electronics includes a wireless transmitter to transmit wireless signal to control at least one of a smartphone, smart speaker, computer or accessibility device to generate the audible spoken word messages.

12. The mask-based diagnostic device of claim 1, wherein the mask-based diagnostic (MBD) system for providing test results using the exhaled breath condensate (EBC) biosample in less than 10 minutes, comprising: the mask having the EBC collector for converting exhaled breath vapor into the collected liquid biosample; the biosensor for generating the output signal dependent on the detection of the target biomarker in the liquid biosample; and electronics for determining the test result from the output signal and controlling the generation of audible spoken word messages for providing automatic readout of at least one of test instructions, test progress and the test results comprise
